(19) United States
(12) Patent Application Publication
(10) Pub. No.: US 2026/0060830 A1
(43) Pub. Date: Mar. 5, 2026

(54) Hinge for Orthopedic Knee Braces with Adjustable Range of Motion

(71) Applicant: Federico Gramaccioni, Ningbo (CN)

(72) Inventor: Federico Gramaccioni, Ningbo (CN)

(21) Appl. No.: 19/275,747

(22) Filed: Jul. 21, 2025

(30) Foreign Application Priority Data
Aug. 28, 2024 (CN) 202422100689.7

(51) Int. Cl. A61F 5/01

(52) U.S. Cl. CPC A61F 5/0123; A61F 2005/0141; A61F 2005/0167

(57) ABSTRACT

A hinge for orthopedic knee braces with adjustable range of rotation, comprises: a first hinge plate (1a), a second hinge plate (1b), two support arms (3) disposed left and right; one or more angle adjustment components (4); wherein a space (10) is defined between the first hinge plate (1a) and the second hinge plate (1b); the heads (3a) of the two support arms (3) are located within the space (10), each head (3a) of each support arm (3) is rotatable and the heads (3a) mesh with each other; each angle adjustment components (4) comprises a retainer frame (41) and a plurality of limiting pins (42), the retainer frame (41) is connected to the second hinge plate (1b) and located above the second hinge plate (1b). In the hinge of the present invention, the angle adjustment components can be mounted on the second hinge plate either before or after the riveting process.

### TECHNICAL FIELD OF THE INVENTION

The present invention relates to a technical field of medical instruments, and in particular to a hinge for orthopedic knee braces with adjustable range of rotation.

### BACKGROUND OF THE INVENTION

Orthopedic braces are worn on the body of a user to stabilize a skeletal joint that has been weakened by injury or other infirmity. Orthopedic braces commonly have rigid structural components that support and stabilize the joint, in order to reduce the compressive force within the part of the knee joint, or to decrease the load on that part of the knee. Additionally, in cases where the knee ligaments are weak and damaged, knee braces can stabilize, protect, support, or rehabilitate the knee.

Generally, the knee brace comprises an upper frame component secured to the upper leg of the user and a lower frame component secured to the lower leg. These frame components are interconnected via a single or double hinge, enabling the brace to follow leg flexion. For example, a U.S. Pat. No. 5,443,444A, titled ORTHOPEDIC POLYCENTRIC HINGE, disclosed a polycentric hinge for orthopedic knee braces, which comprises two pair of rigid support arms having cooperating, inter locking proximal ends, a pair of pivot pins and a hinge plate. The arms are rotatably coupled at their proximal ends to the hinge plate through the pivot pins and are rotatable between an adjustable extension position and an adjustable flexion position. The hinge plate has a plurality of adjustment apertures therein adapted for selective reception of an extension limit pin and a flexion limit pin to selectively restrain rotation of the arms beyond a final extension position and a final flexion position. The pins are secured to the hinge assembly by elastomeric tethers and are positionable between the apertures to selectively adjust the range of motion of the arms.

However, in the aforementioned solution, the angle adjustment component is fixedly connected to the hinge upper plate during the riveting process, and cannot be assembled or replaced after the hinge assembled together.

### SUMMARY OF THE INVENTION

It is an object of the present invention to provide a hinge for orthopedic knee braces with adjustable range of rotation, allowing installation of the angle adjustment component either before or after the riveting process.

For achieving the above object, the hinge for orthopedic knee braces with adjustable range of rotation comprises:
an upper and a lower transverse hinge plate, separated from each other;
two support arms disposed left and right, each having a head;
one or more angle adjustment components;
wherein, the lower hinge plate is defined as a first hinge plate, the upper hinge plate is defined as a second hinge plate, a space is defined between the first hinge plate and the second hinge plate;
the heads of the two support arms are located within the space and are disposed face to face, each head of each support arm is rotatable and the heads mesh with each other;
each angle adjustment component comprises a retainer frame and a plurality of limiting pins each disposed on the retainer frame and extending into the space, and the retainer frame is connected to the second hinge plate and located above the second hinge plate.

To adjust the mounting positions of the limiting pins, preferably, the first hinge plate has two groups of first adjustment holes, each group of first adjustments holes corresponding to one support arm, and each group of first adjustment holes comprises a plurality of first adjustment holes disposed at intervals along a rotational direction of the corresponding support arm;
the second hinge plate has two groups of second adjustment holes, each group of second adjustment holes corresponding to one group of first adjustment holes, and each group of second adjustment holes comprises a plurality of second adjustment holes corresponding to the plurality of first adjustment holes;
two angle adjustment components are disposed left and right above the second hinge plate, each angle adjustment component corresponds to one support arm;
each angle adjustment component comprises two limiting pins each having a top end and a bottom end, the top end of each limiting pin is connected to the corresponding retainer frame, and the bottom end of each limiting pin passes through one of the second adjustment holes in the corresponding group of second adjustment holes and the space and is then inserted into a first adjustment hole in the corresponding group of first adjustment holes; when the two limiting pins of each angle adjustment component pass through the space, the two limiting pins are located at a side of the corresponding support arm, so that the support arm is capable of resisting against the two limiting pins during the rotation of the support arm so as to limit a rotational range of the corresponding support arm.

To facilitate the flexible movement of the limiting pins, preferably, the retainer frame comprises a base connected to the second hinge plate and two flexible arms extending outward from the base, each limiting pin corresponds to one flexible arm, and a head of each limiting pin is connected to a free end of the corresponding flexible arm.

To realize the snap-fit connection of the base to the second hinge plate, preferably, the base has two third elastic clips extending downward from a bottom of the base, the second hinge plate has two latch holes corresponding to the third elastic clips, and each third elastic clip is latched within the corresponding latch hole so as to connect the base to the second hinge plate.

To simplify the assembly process of the angle adjustment components, preferably, the two bases in two angle adjustment components are integrally connected to each other.

To facilitate the assembly of the angle adjustment components, preferably, each flexible arm is integrally formed on the base, and the head of each limiting pin inserts inside the free end of the corresponding flexible arm.

To improve the mounting stability of the limiting pins without affecting the movement of the limiting pins, preferably, each flexible arm is an S-shaped elastic component.

To protect the angle adjustment components from falling off, preferably, the hinge further comprises a top cover disposed above the angle adjustment components, a rear side of the top cover is hinged to the second hinge plate, and a front side of the top cover is detachably connected to the first hinge plate.

To ensure the low friction coefficient and high mechanism strength of the hinge plates, preferably, each hinge plate comprises a plastic plate and a metal plate molded inside the plastic plate.

To facilitate the rotatable connection of the support arms to the hinge plates, the hinge further comprises two rivets each having a head and a tail corresponding to the support arms, the first hinge plate has two mounting holes corresponding to the rivets vertically penetrating through the first hinge plate, and each support arm has a pivot hole vertically penetrating through the head of each support arm; and
the head of each rivet is molded inside the plastic plate of the second hinge plate in an insert manner, and the tail of each rivet passes through the corresponding pivot hole and mounting hole and is then anchored onto the first hinge plate.

Compared with the prior art, the present invention has the following advantages. Since the retainer frame of the angle adjustment components is connected to the second hinge plate in a snap-fit manner and located above the second hinge plate, the angle adjustment components can be mounted to the second hinge plate either before or after the riveting process, it makes the assembly operation more easy and convenient, and it is convenient for replacement of the angle adjustment components.

### BRIEF DESCRIPTION OF THE DRAWINGS

FIG. 1 is a perspective view of a hinge for orthopedic knee braces according to an embodiment of the present invention;

FIG. 2 is a perspective view of FIG. 1 (omitting the rivets);

FIG. 3 is a sectional view of line A-A of FIG. 1;

FIG. 4 is a sectional view of line B-B of FIG. 1;

FIG. 5 is a sectional view of line C-C of FIG. 1.

### DETAILED DESCRIPTION OF THE INVENTION

The present invention will be further described below in detail by embodiments with reference to the accompanying drawings.

It should be noted that in the description and claims of the present invention, the terms used to indicate direction, such as “front, back”, “up, down”, “left, right”, “side, top, bottom”, etc. are used to describe structures and elements of the present invention for better explanation. And these terms used here are based on an orientation in the accompanying drawings. Since the embodiments disclosed by the present invention can be set in different directions, these terms indicating directions are only used as explanations and should not be used as restrictions. For example, the verbs “up”, “down” should not be limited to the direction opposite or consistent with the gravity.

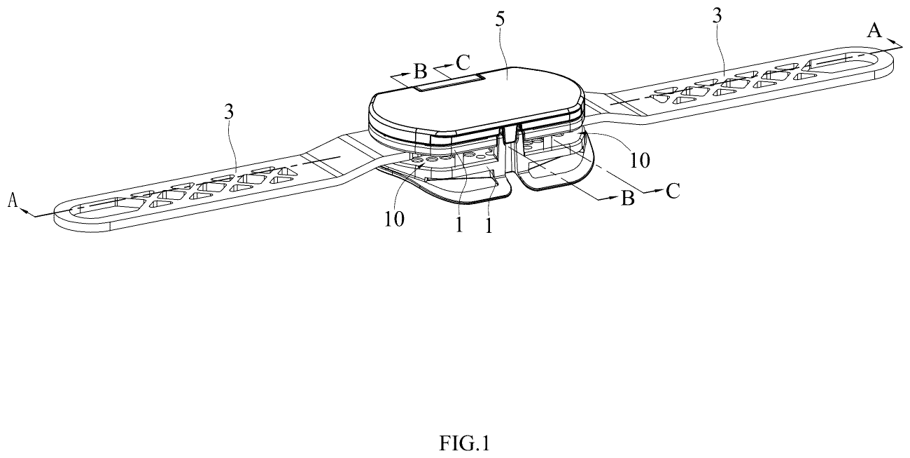

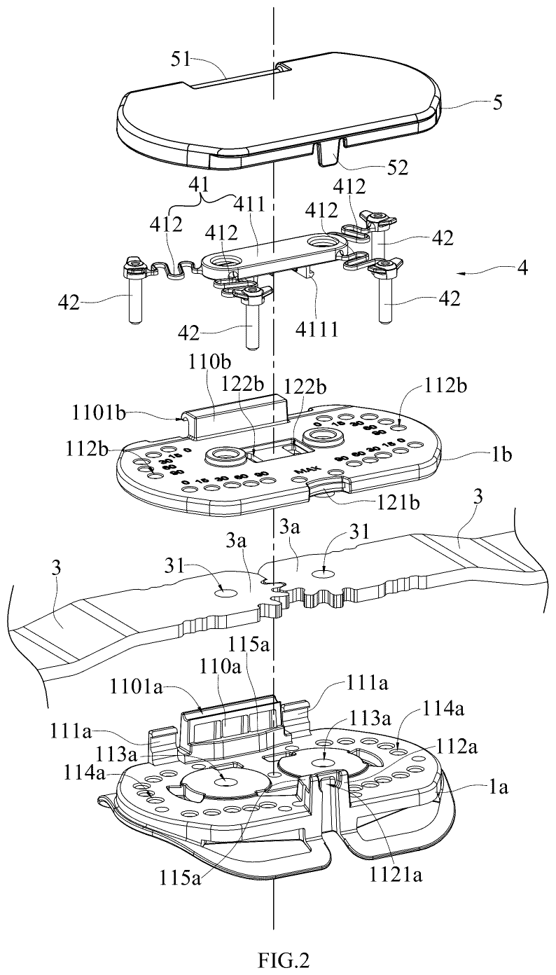

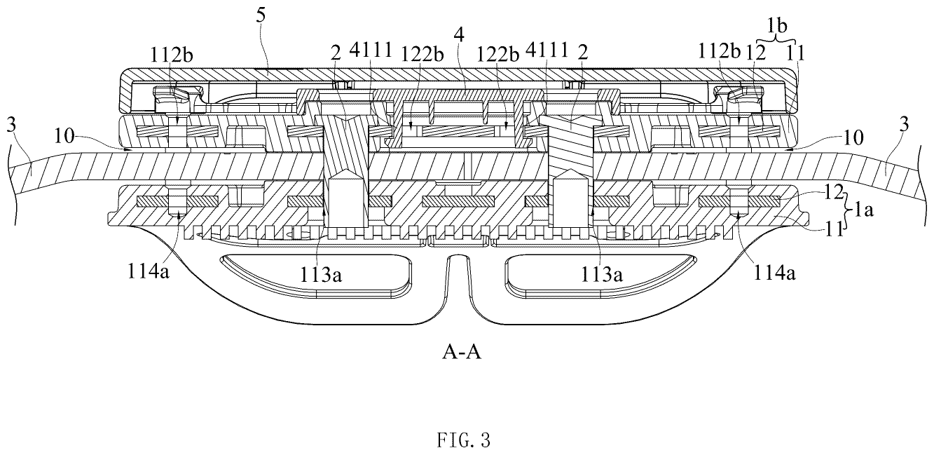

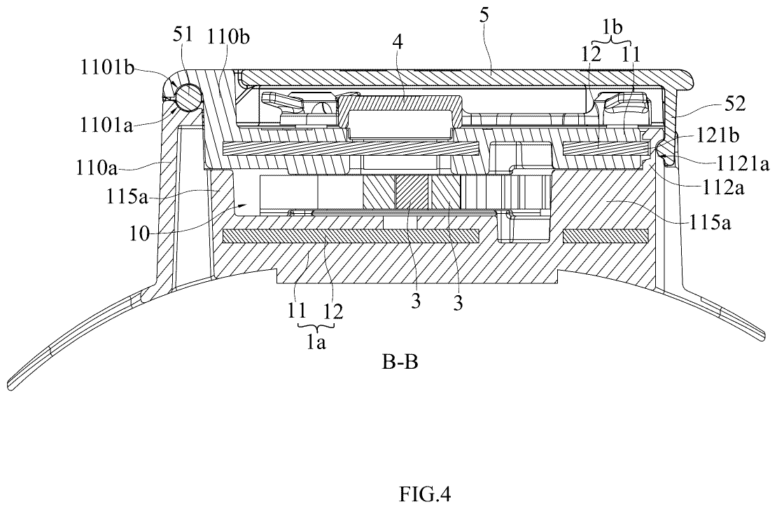

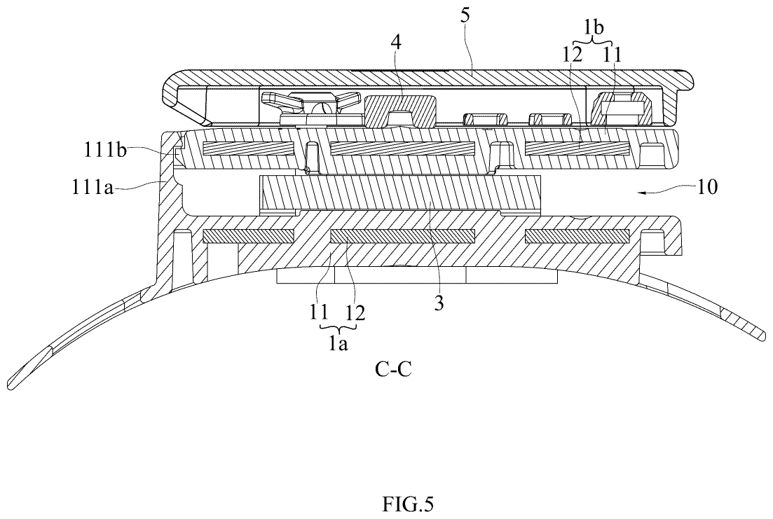

FIGS. 1-5 show a preferred embodiment of the hinge for an orthopedic knee brace of the present invention. The hinge comprises two hinge plates 1, two rivets 2, two support arms 3, two angle adjustment components 4 and a top cover 5.

The hinge has an upper and a lower transverse hinge plate 1 separated from each other, the lower hinge plate is defined as a first hinge plate 1a, the upper hinge plate is defined as a second hinge plate 1b, a space 10 is defined between the first hinge plate 1a and the second hinge plate 1b, and the second hinge plate 1b is connected to the first hinge plate 1a in a snap-fit manner, finally the second hinge plate 1b and the first hinge plate 1a are connected together through one or more fasteners.

In this embodiment, each hinge plate 1 comprises a plastic plate 11 and a metal plate 12 molded inside the plastic plate 11, thus simultaneously ensuring the low friction coefficient and high mechanical strength of the hinge plate 1.

Specifically, in the first hinge plate 1a, the plastic plate 11 has a first hinge seat 110a extending upward and two first elastic clips 111a on a rear side of the plastic plate 11, the two first elastic clips 111a are located on the left and right sides of the first hinge seat 110a respectively, and the plastic plate 11 has a second elastic clip 112a extending upward on a front side of the plastic plate 11. A first hole slot 1101a extending laterally and opened upward is formed on the top of the first hinge seat 110a. The first elastic clips 111a are arranged opposite to a head (as a snap-fit portion) of the second elastic clip 112a, and the second elastic clip 112a has a latch slot 1121a opened forward. The first hinge plate 1a has two laterally spaced mounting holes 113a vertically arranged. The first hinge plate 1a also has two groups of first adjustment holes, each group of first adjustments holes corresponding to the mounting holes 113a, and each group of first adjustment holes comprises a plurality of first adjustment holes 114a disposed at intervals along a circumferential direction of the corresponding mounting holes 113a. In addition, the plastic plate 11 has two bosses 115a for supporting the second hinge plate 1b protruding upward, respectively on a front side of the first hinge seat 110a and a rear side of the second elastic clip 112a.

In the second hinge plate 1b, the plastic plate 11 has an inverted L-shaped second hinge seat 110b protruding upward on a rear side of the plastic plate 11, and a second hole slot 1101b extending laterally and opened downward formed on a top of the second hinge seat 110b. The plastic plate 11 has two first flanges 111b extending backward and each correspondence to one first elastic clip 111a on a rear side of the plastic plate 11, and a head (as a snap-fit portion) of each first elastic clip 111a is positioned (snap-fitted) on the corresponding first flange 111b. The metal plate 12 has a second flange 121b extending forward and extending out of the plastic plate 11 on a front side of the metal plate 12, and a head (as a snap-fit portion) of the second elastic clip 112a is positioned (snap-fitted) on the second flange 121b. The second hinge plate 1b has two groups of second adjustment holes, each group of second adjustment holes corresponding to one group of first adjustment holes, and each group of second adjustment holes comprises a plurality of second adjustment holes 112b corresponding to the plurality of first adjustment holes 114a. And, the second hinge plate 1b has two laterally spaced latch holes 122b.

Of course, instead of the first flanges 111b and the second flange 121b, it is also possible to provide latch slots, latch holes or other structures as snap-fit portions on the second hinge plate 1b. It is also possible to interchange the positions of the elastic clips and the snap-fit portions on the first hinge plate 1a and the second hinge plate 1b.

There are two rivets 2 which are corresponding to the two mounting holes 113a each having a head and a tail, the head of each rivet 2 is molded inside the plastic plate 11 of the second hinge plate 1b in an insert manner, and the tail of each rivet 2 extends out of the bottom of the plastic plate 11 of the second hinge plate 1b.

There are two support arms 3 which are corresponding to the two rivets 2 and disposed left and right, each having a head, and the heads of the two support arms 3 are located within the space 10 and meshed with each other. Specifically, two pivot holes 31 are vertically penetrated through on the head of each support arm 3, and the tail of each rivet 2 passes through the corresponding pivot hole 31 and mounting hole 113a and is then anchored onto the first hinge plate 1a, so that each support arm 3 can rotate about the corresponding rivet 2 relative to the hinge plate 1.

There are two angle adjustment components 4 which are corresponding to the two support arms 3. Each angle adjustment component 4 is mounted above the second hinge plate 1b to limit the rotation angle of the corresponding support arm 3. In this embodiment, each angle adjustment component 4 comprises a retainer frame 41 and two limiting pins 42.

Specifically, the retainer frame 41 comprises a base 411 and two flexible arms 412 extending outward from the base 411. In this embodiment, the two bases 411 in two angle adjustment components 4 are integrally connected to each other, the base 411 has two third elastic clips 4111 extending downward from a bottom of the base 411, the second hinge plate 1b has two latch holes 122b corresponding to the third elastic clips 4111, and each third elastic clip 4111 is latched within the corresponding latch hole 122b so as to connect the base 411 to the second hinge plate 1b. It is also possible to interchange the positions of the elastic clips and the latch holes on the base 411 and the second hinge plate 1b; and each flexible arm 412 is integrally formed on the base 411.

There are two limiting pins 42 which are in one-to-one correspondence to the flexible arms 412, and the head of each limiting pin 42 inserts inside the free end of the corresponding flexible arm 412, and the bottom end of each limiting pin 42 passes through one of the second adjustment holes 112b in the corresponding group of second adjustment holes and the space 10 and is then inserted into a first adjustment hole 114a in the corresponding group of first adjustment holes, so as to limit a rotational range of the corresponding support arm 3.

In this embodiment, each flexible arm 412 is an S-shaped elastic component in order to facilitate the position adjustment of the limiting pins 42.

A top cover 5 is disposed above the angle adjustment components 4. Specifically, a rotating shaft 51 extending laterally is provided on the rear side of the top cover 5, and the rotating shaft 51 passes through the pivot hole formed by the first hole slot 1101a and the second hole slot 1101b in a rotatable manner, so as to hinge the rear side of the top cover 5 to the second hinge plate 1b; and, a fourth elastic clip 52 extending downward is provided on the front side of the top cover 5, and the fourth elastic clip 52 is latched within the latch slot 1121a, so as to realize the detachably connection of the front side of the top cover 5 to the first hinge plate 1a.

During mounting:
(1) The top cover 5 is placed above the second hinge plate 1b, and the rotating shaft 51 of the top cover 5 is inserted into the second hole slot 1101b on the second hinge plate 1b, so as to realize pre-positioning the top cover 5 on the second hinge plate 1b;
(2) Then, the two rivets 2 on the second hinge plate 1b are sequentially inserted downward into the pivot holes 31 on the corresponding support arms 3 and the mounting holes 113a on the first hinge plate 1a until the snap-fit portions of the heads of the first elastic clips 111a are snap-fitted on the corresponding first flanges 111b, and the snap-fit portion of the head of the second elastic clip 112 is snap-fitted on the second flange 121b, thereby realizing pre-positioning between the first hinge plate 1a and the second hinge plate 1b;

Meanwhile, the rotating hole 51 of the top cover 5 is limited within the pivot hole formed by the first hole slot 1101a and the second hole slot 1101b, and the fourth elastic clip 52 of the top cover 5 is latched within the latch slot 1121a on the first hinge plate 1a.
(3) Then, each rivet 2 is anchored onto the first hinge plate 1a to assemble the two hinge plates 1, the two support arms 3 and the top cover 5 together.
(4) Finally, the top cover 5 is turned upside down, the third elastic clips 4111 of the angle adjustment components 4 are clamped downward into the latch slots 122b on the second hinge plate 1b, and the tails of the limiting pins 42 are inserted into the corresponding second adjustment holes 112b in place, so that the whole hinge for orthopedic knee braces is mounted.

The present invention has the following advantages:

Firstly, the second hinge plate 1b is connected to the first hinge plate 1a in a snap-fit manner to realize pre-positioning between the first hinge plate 1a and the second hinge plate 1b without aligning and limiting the both during anchoring, achieving easy components.

Secondly, since the rivets 2 are pre-arranged on the second hinge plate 1b, the rivets 2 can be arranged during the snap-fit connection of the first hinge plate 1a to the second hinge plate 1b. This simplifies the components process.

Thirdly, since the head of each limiting pin 42 inserts inside the free end of the corresponding flexible arm 412, the retainer 4 and the limiting pins 42 do not need to be assembled in the assembly process, achieving easy assembly.

Fourthly, since the two bases 411 in two angle adjustment components 4 are integrally connected to each other, the angle adjustment components 4 only need to be mounted at one time, thereby simplifying the assembly process.

Fifthly, the angle adjustment components 4 can be mounted to the second hinge plate 1b either before or after the riveting process, achieving flexible assembly and convenient replacement of the angle adjustment components 4.

1. A hinge for orthopedic knee braces with adjustable range of rotation, comprising:
an upper and a lower transverse hinge plate (1) separated from each other;
two support arms (3) disposed left and right, each having a head (3a);
one or more angle adjustment components (4);
wherein, the lower hinge plate is defined as a first hinge plate (1a), the upper hinge plate is defined as a second hinge plate (1b), a space (10) is defined between the first hinge plate (1a) and the second hinge plate (1b);
the heads (3a) of the two support arms (3) are located within the space (10) and are disposed face to face, each head (3a) of each support arm (3) is rotatable and the heads (3a) mesh with each other;
each angle adjustment components (4) comprises a retainer frame (41) and a plurality of limiting pins (42) each disposed on the retainer frame (41) and extending into the space (10), and the retainer frame (41) is connected to the second hinge plate (1b) and located above the second hinge plate (1b).

2. The hinge of claim 1, wherein the first hinge plate (1a) has two groups of first adjustment holes, each group of first adjustments holes corresponding to one support arm (3), and each group of first adjustment holes comprises a plurality of first adjustment holes (114a) disposed at intervals along a rotational direction of the corresponding support arm (3);
the second hinge plate (1b) has two groups of second adjustment holes, each group of second adjustment holes corresponding to one group of first adjustment holes, and each group of second adjustment holes comprises a plurality of second adjustment holes (112b) corresponding to the plurality of first adjustment holes (114a);
two angle adjustment components (4) are disposed left and right above the second hinge plate (1b), each angle adjustment component (4) corresponds to one support arm (3);
each angle adjustment component (4) comprises two limiting pins (42) each having a top end and a bottom end, the top end of each limiting pin (42) is connected to the corresponding retainer frame (41), and the bottom end of each limiting pin (42) passes through one of the second adjustment holes (112b) in the corresponding group of second adjustment holes and the space (10) and is then inserted into a first adjustment hole (114a) in the corresponding group of first adjustment holes;
when the two limiting pins (42) of each angle adjustment component (4) pass through the space (10), the two limiting pins (42) are located at a side of the corresponding support arm (3), so that the support arm (3) is capable of resisting against the two limiting pins (42) during the rotation of the support arm (3) so as to limit a rotational range of the corresponding support arm (3).

3. The hinge of claim 2, wherein the retainer frame (41) comprises a base (411) connected to the second hinge plate (1b) and two flexible arms (412) extending outward from the base (411), each limiting pin (42) corresponds to one flexible arm (412), and a head of each limiting pin (42) is connected to a free end of the corresponding flexible arm (412).

4. The hinge of claim 3, wherein the base (411) has two third elastic clips (4111) extending downward from a bottom of the base (411), the second hinge plate (1b) has two latch holes (122b) corresponding to the third elastic clips (4111), and each third elastic clip (4111) is latched within the corresponding latch hole (122b) so as to connect the base (411) to the second hinge plate (1b).

5. The hinge of claim 3, wherein the two bases (411) in two angle adjustment components (4) are integrally connected to each other.

6. The hinge of claim 3, wherein each flexible arm (412) is integrally formed on the base (411).

7. The hinge of claim 3, wherein each flexible arm (412) is an S-shaped elastic component.

8. The hinge of claim 1, further comprising a top cover (5) disposed above the angle adjustment components (4), a rear side of the top cover (5) is hinged to the second hinge plate (1b), and a front side of the top cover (5) is detachably connected to the first hinge plate (1a).

9. The hinge of claim 1, wherein each hinge plate (1) comprises a plastic plate (11) and a metal plate (12) molded inside the plastic plate (11).

10. The hinge of claim 9, further comprising two rivets (2) each having a head and a tail corresponding to the support arms (3), the first hinge plate (1a) has two mounting holes (31) corresponding to the rivets (2) vertically penetrating through the first hinge plate (1a), and each support arm (3) has a pivot hole (113a) vertically penetrating through the head of each support arm (3); and
the head of each rivet (2) is molded inside the plastic plate (11) of the second hinge plate (1b) in an insert manner, and the tail of each rivet (2) passes through the corresponding pivot hole (31) and mounting hole (113a) and is then anchored onto the first hinge plate (1a).
